(19) United States
(12) Patent Application Publication
(10) Pub. No.: US 2019/0209838 A1
(43) Pub. Date: Jul. 11, 2019

(54) ELECTRODES FOR USE IN ELECTRICAL STIMULATION DEVICES AND ASSOCIATED METHODS

(71) Applicant: ReliefBand Technologies, LLC, Horsham, PA (US)

(72) Inventors: Nicholas Spring, Jenkintown, PA (US); Jacqueline Simon, New Hope, PA (US)

(21) Appl. No.: 16/241,003

(22) Filed: Jan. 7, 2019

Related U.S. Application Data
(60) Provisional application No. 62/614,212, filed on Jan. 5, 2018.
(60) Provisional application No. 62/614,211, filed on Jan. 5, 2018.

(51) Int. Cl. A61N 1/36; A61N 1/375; A61N 1/04; A61H 39/00

(52) U.S. Cl. CPC A61N 1/36014; A61H 39/002; A61N 1/0408; A61N 1/375

(57) ABSTRACT

A P6 electrostimulation device has a pair of electrodes connected to electronic control circuitry that includes a power supply and pulse generation circuitry. The electrodes of the device are made of 316L stainless steel and manufactured by metal injection molding. Each electrode may be a D shaped electrode made of 316L stainless steel and manufactured by metal injection molding. The electrode has a straight edge having a length of about 22.00 to about 25.50 mm and an arcuate edge having an inner radius of about 10.50 to about 13.00 mm, and a thickness of about 0.50 to about 1.75 mm.

### CROSS-REFERENCE TO RELATED APPLICATIONS

The present application claims priority to U.S. Provisional Patent Application Ser. No. 62/614,211, filed on Jan. 5, 2018, and U.S. Provisional Patent Application Ser. No. 62/614,212, filed on Jan. 5, 2018, the disclosures of which are hereby incorporated in their entirety at least by reference.

### FIELD OF THE INVENTION

The devices and device components described below and methods of using the same relate to the fields of electrodes used in noninvasive electrical stimulation of an acupuncture point.

### BACKGROUND OF THE INVENTION

Bertolucci, Nausea Control Device, U.S. Pat. No. 4,981,146, Jan. 1, 1991, which is incorporated herein by reference, describes a nausea control device in the form of a watch-like housing attachable to the human wrist by an adjustable attachment band. The device uses non-invasive nerve stimulation whereby electricity is passed through two electrodes to stimulate nerves located on the ventral side of the wrist (this anatomical position is sometimes referred to as the palmar side of the wrist). The ventral site of application is referred to in the acupuncture art as the P6 point, pericardium 6 point, or master point of the pericardium meridian (sometimes referred to as the vascular meridian). A primary object of the Bertolucci device is to provide a non-chemical, non-invasive, painless and inexpensive method of alleviating nausea. It is also portable, self-contained and convenient to the patient. Electrical pulse repetition rate of approximately 70 pulses per second and a pulse width of 80 microseconds has been found to provide effective relief of nausea in a patient. Electrical pulse pattern comprises about 350 microsecond pulse width at about 31 pulses per second at power levels of about 10-40 milliamps peak pulse height is disclosed. A wide range of pulse patterns may be used in non-invasive nerve stimulation devices. Nausea control devices like those described in U.S. Pat. No. 4,981,146 have been sold to treat nausea caused by chemotherapy, motion sickness and morning sickness associated with pregnancy and used as an adjunct to antiemetics therapy for reducing post-operative nausea, retching and vomiting. Similar devices have been disclosed for use in treating dysmenorrhea and menstrual cramps (U.S. Pat. Nos. 6,282,443 and 6,718,202, which are each incorporated herein by reference), for use in moderating blood pressure (U.S. Pat. Nos. 6,178,352 and 6,658,298, which are each incorporated herein by reference) and for reducing gag reflex (U.S. Pat. No. 6,192,889, which is incorporated herein by reference).

U.S. Pat. Nos. 6,735,480 and 7,983,761, which are incorporated herein by reference disclose devices for delivering electrostimulation to the P6 point using electrode assembly designs. Among the design features described in these patents, electrodes are described as a pair of metal electrodes that are substantially D-shaped, i.e. having a straight edge and an arcuate edge. The electrodes are arranged such that when the device is properly worn by a user and the electrodes are in contact with the P6 point on a user's wrist the straight edges of the electrodes oppose each other. The straight edges are thus parallel to each other. The electrodes are arranged on the device such that when the device is properly worn by a user and the electrodes are in contact with the P6 point on a user's wrist the parallel, the straight edges of the electrodes are perpendicular with the direction of the users arm between the wrist and elbow. The straight edges have a length of about 1 inch, and the arcuate edges having an inner radius of about 0.5 inches. The electrodes found in these device are made from metal and are typically formed using a stamping operation and suffer from some drawbacks in that the material and shape of the electrode is limited by the stamping operation. The present disclosure is directed to providing overcoming one or more drawbacks associated with previous electrodes.

### SUMMARY OF THE INVENTION

The electrodes described herein may be used in devices such as those described in U.S. Pat. Nos. 4,981,146, 6,076,018, 6,567,695, 6,735,480, 7,127,288, 7,171,266, 7,171,276, 7,983,761 which are each incorporated herein by reference, and similar devices. Devices comprising the electrodes may be used in methods to prevent and treat nausea caused by chemotherapy, motion sickness and morning sickness associated with pregnancy and used as an adjunct to antiemetics therapy for reducing post-operative nausea, retching and vomiting. Devices comprising the electrodes may also be used in methods to prevent and treat dysmenorrhea and menstrual cramps, and to moderate blood pressure. In addition, devices comprising the electrodes may also be used in methods to treat alcohol hangover, and to prevent or treat vertigo symptoms and virtual reality sickness. The electrodes are made of 316L stainless steel manufactured using metal injection molding technology.

A P6 electrostimulation device including a pair of electrodes connected to electronic control circuitry that comprises a power supply and pulse generation circuitry is provided. The electrodes of the device are made of 316L stainless steel and manufactured by metal injection molding.

A D shaped electrode made of 316L stainless steel and manufactured by metal injection molding is provided. The electrode has a straight edge having a length of about 22.00 to about 25.50 mm and an arcuate edge having an inner radius of about 10.50 to about 13.00 mm, and a thickness of about 0.50 to about 1.75 mm.

### BRIEF DESCRIPTION OF THE FIGURES

FIGS. 1A, 1B, and 1C show side and perspective views of an embodiment of a P6 electro-stimulation device which includes electrodes;

FIG. 2 is a perspective view of an exemplary electrode;

FIG. 3 includes a variety of views of the exemplary electrode; and

FIG. 4 further illustrates an exemplary electrode and a corresponding electrical conduit which may be used in conjunction with the electrode.

### DETAILED DESCRIPTION OF PREFERRED EMBODIMENTS

Electrodes which are used in P6 electrostimulation devices and are made of 316L stainless steel manufactured using metal injection molding technology have been found to provide more comfort and less incidence of painful incidents in use compared to devices made using electrodes used in previous devices that were made from different material stamped from rolled sheets of metal. The problems associated with the discomfort and occasional pain experience using the electrodes of previous P6 electrostimulation devices result in the need to reduce the power output when using the device to minimize discomfort and incidence of pain. Providing electrodes made of 316L stainless steel manufactured using metal injection molding technology addresses this problem by promoting a more tolerable sensation which allows the device to be used by more people. Moreover, the metal injection molding process may result in smaller and more homogeneous grain structure in the electrodes, which produces a better and more even distribution of current during therapy delivery. P6 electrostimulation devices that comprise electrodes made of 316L stainless steel manufactured using metal injection molding technology thus may be more effective compared to previous comparable devices.

Metal injection molding technology provides processes for injection molding articles from metal powders including 316L stainless steel powder. Examples of metal injection molding technology are described in U.S. Pat. Nos. 4,113,480, 4,227,926, 4,305,757, 4,404,166, 4,478,790, 5,059,388, 5,985,208, 6,056,915 and 6,846,862, and U.S. Patent Application Nos. US20080015683, US20060276875, which are incorporated herein by reference.

Electrodes made of 316L stainless steel manufactured using metal injection molding technology in P6 electrostimulation devices are provided as the electrodes in devices such as those described in U.S. Pat. Nos. 4,981,146, 6,076,018, 6,567,695, 6,735,480, 7,127,288, 7,171,266, 7,171,276, 7,983,761, and provisional application no #### entitled WEARABLE DEVICE FOR DELIVERING ELECTRICAL STIMULATION TO THE P6 ACUPUNCTURE POINT and filed Jan. 5, 2017.

In a preferred embodiment, the P6 electrostimulation devices comprises a pair of metal electrodes made of 316L stainless steel manufactured using metal injection molding technology that are substantially D-shaped, i.e. having a straight edge and an arcuate edge. The D shaped electrodes made of 316L stainless steel manufactured using metal injection molding technology are arranged such that when the device is properly worn by a user and the electrodes are in contact with the P6 point on a user's wrist, the straight edges of the electrodes oppose each other, i.e. arranged with the straight edges facing each other in apposition. The straight edges are thus parallel to each other. The electrodes are arranged on the device such that when the device is properly worn by a user and the electrodes are in contact with the P6 point on a user's wrist, the straight edges are perpendicular with the direction of the user's arm between the wrist and elbow. The straight edges have a length of about 1 inch, and the arcuate edges having an inner radius of about 0.5 inches. In a preferred embodiment, each D shaped electrode is about 22.00 to about 25.50 mm and the arcuate edges having an inner radius of about 10.50 to about 13.00 mm. In a preferred embodiment, each D shaped electrode has a thickness of about 0.50 to about 1.75 mm. In a preferred embodiment, each D shaped electrode is about 23.00 to about 24.50 mm and the arcuate edges having an inner radius of about 11.00 to about 12.70 mm. In a preferred embodiment, each D shaped electrode has a thickness of about 0.80 to about 1.40 mm. In a preferred embodiment, D shaped electrode is about 23.84+/−0.10 mm and the arcuate edges having an inner radius of about 11.83+/−0.10 mm. In a preferred embodiment, each D shaped electrode has a thickness of about 1.08+/−0.10 mm.

The electrodes are arranged on the device such that the straight edges of the electrodes face each other in apposition and are separated by a gap.

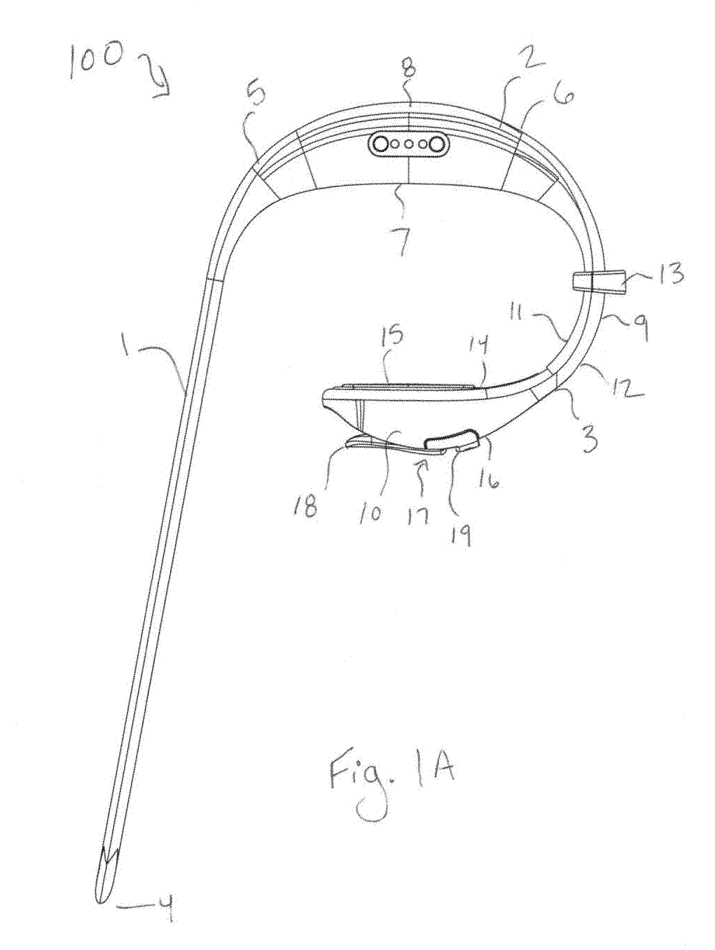

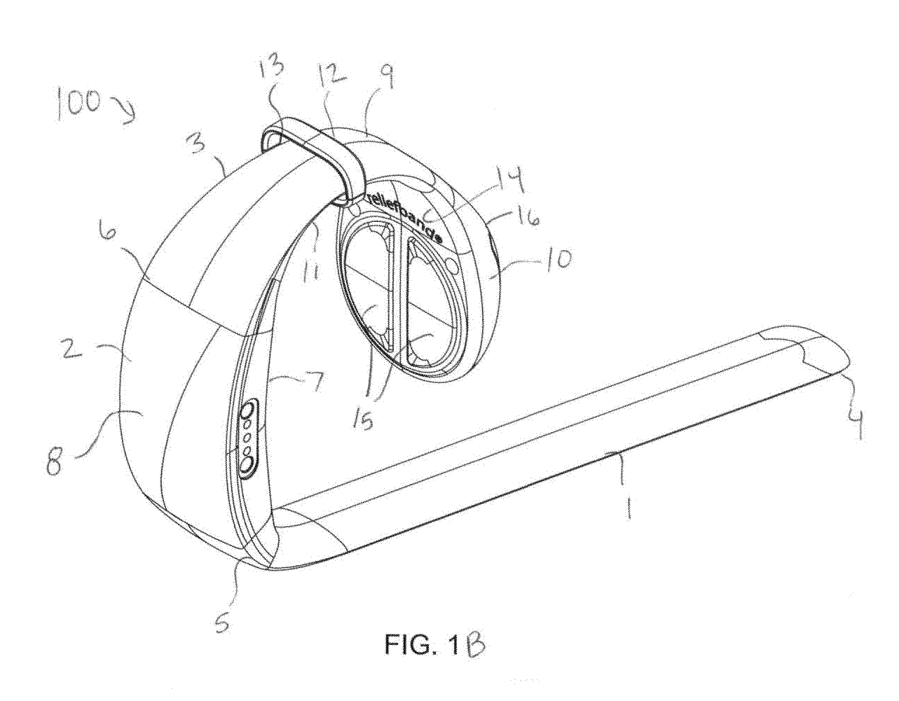

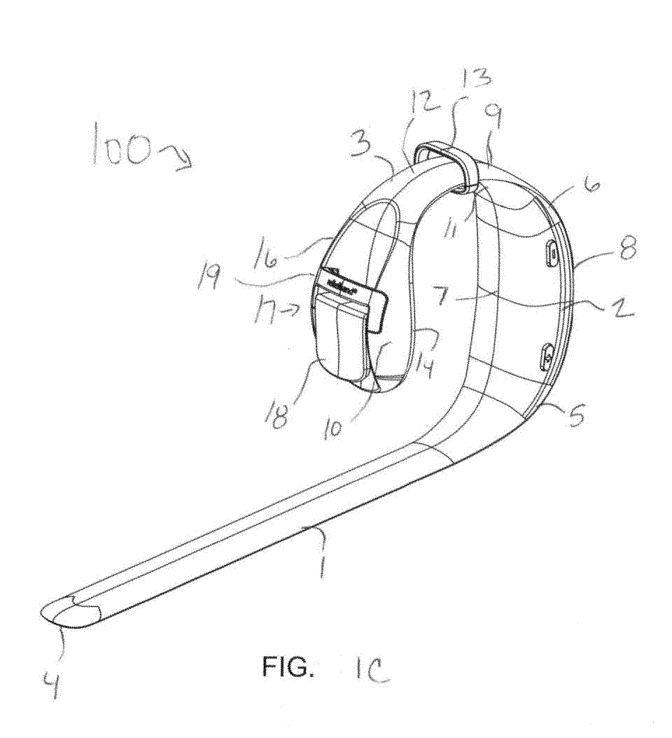

FIG. 1A shows a side view of an embodiment of an exemplary P6 electro-stimulation device 100 which may include electrodes consistent with the present disclosure. FIGS. 1B and 1C show perspective views of the device 100, further illustrating features described below. Three main sections of the device 100 are shown: a simple band 1, a user interface housing 2 and a J-band 3. The terms simple band and J-band are used for the sake of convenience and are not limited to simple and/or J-band components. For example, the simple band 1 may be a first band which forms a first portion of the device 100 and the J-band 3 a second band which forms a second portion of the device 100. Together with the interface housing 2, these first and second bands form an enclosed space for receiving the wrist of the user.

The simple band 1 has a free end 4. The simple band 1 and the user interface housing 2 are attached to each other at the simple band-user interface housing attachment point 5. The user interface housing 2 is attached to the J-band 3 at the J-band-user interface housing attachment point 6. The user interface housing 2 has an inner dorsum surface 7 and an outer display surface 8 and preferably a slight curve for better fit when in contact with the dorsum side of the user's wrist. The J-band 3 has a flexibly rigid curved section 9 and a palmar section 10. The flexibly rigid curved section 9 has inside curved surface 11, an outside curved surface 12 and a strap loop 13. The palmar section 10 comprises the flat inner surface 14 that comprises a pair of electrodes 15 separated by a gap (shown in FIG. 1B) and outer surface 16 comprising a clasp assembly 17 that comprises a cam 18 and a cinch bar 19. Without a load on the flexibly rigid curved section 9, the flat inner surface 14 of the palmar section 10 of the J-band 3 faces the inner dorsum surface 7 of the user interface housing 2.

In some embodiments, the gap between the electrodes 15 is 0.05 to 0.5 inches or 1-15 mm. In some embodiments, the gap is 0.1 to 0.25 inches or 2-7.5 mm. In some embodiments, the gap is 03-5 mm. In other embodiments, the gap is 0.14 inches.

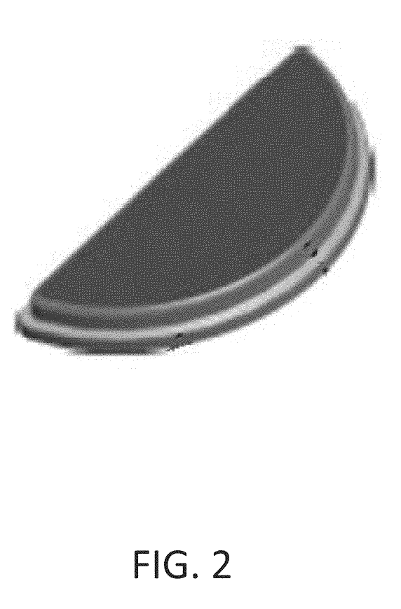

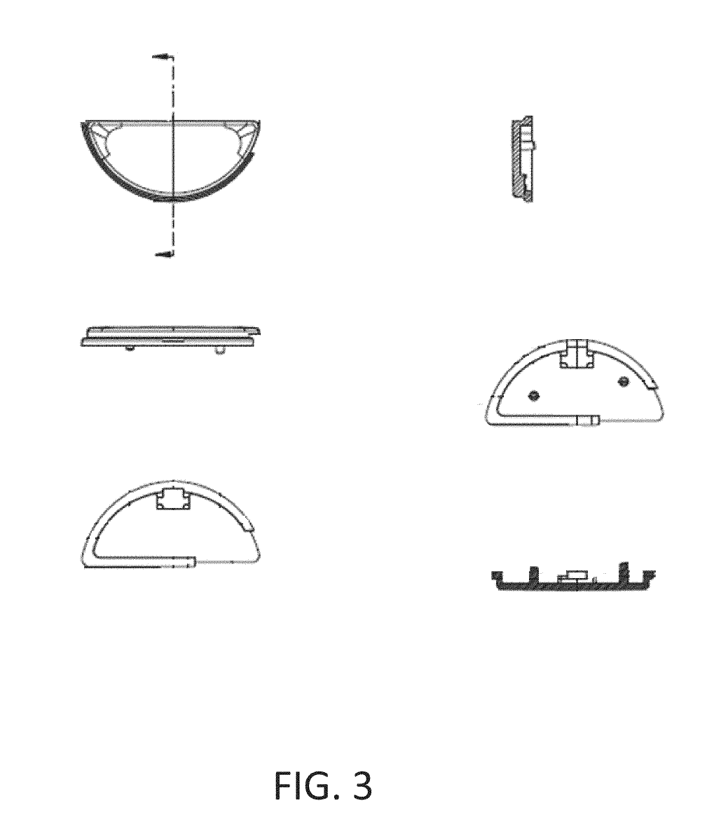

FIGS. 2 and 3 further illustrate an exemplary embodiment of one of the electrodes 15. The electrodes 15 may include a first surface for contacting the user and an opposite surface for attachment to the device. The first surface may include a beveled edge. The opposite surface may include a concave portion. The electrode 15 is preferably made of 316L stainless steel and manufactured by metal injection molding to obtain the illustrated shape and configuration. The illustrated configuration of the electrode 15 is exemplary and that other shapes and configurations are possible. The material and injection molding process enable the electrodes 15 to be custom formed to fit a particular device configuration, such as the J-shaped band 3 of the device 100.

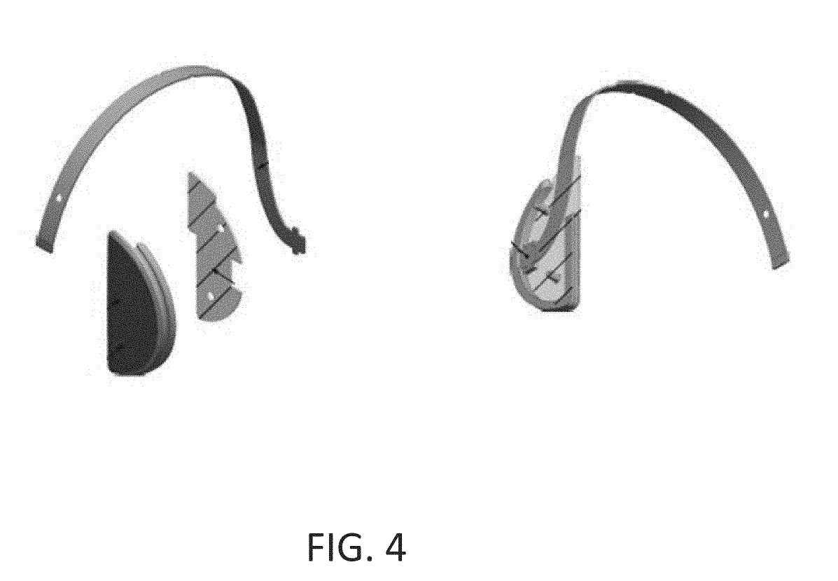

FIG. 4 shows attachment of an electrode to an electrical conduit. The electrical conduit is attached to the electrode by an electrode fastener tab. The electrical conduit is preferably formed in a curved shape that matches the flexibly rigid curved section 9 such that the electrical conduit may traverse from the electrodes to the user interface housing. The user interface housing preferably includes electronics which connect to the electrical conduit.

In some embodiments, the D shaped electrodes are used in the P6 electrostimulation devices and the electrodes are connected to the electrical conduits that supply electrical output at a position on or immediately adjacent to, i.e. within 5 mm, preferably within 3 mm of the center point of the arcuate edge.

In some embodiments, the P6 electrostimulation device provided herein the pulse generation (output) of the device is as follows. Waveform may be asymmetrical balanced and biphasic. In some embodiments, the power level of the output is from 0.1 mA to 50 mA. In some embodiments, the power level of the output is from <1 mA to 40 mA. In some embodiments, ten (10) intensity levels (defined by the maximum current amplitude measured at the peak of the stimulus pulse when unit is driving a 500 ohm purely resistive load) will be available (all expressed as “setting level=output in milliamperes”) are provided. In some embodiments, the ten intensity settings may be as follows: 1=8, 2=11, 3=14, 4=17, 5=20, 6=24, 7=28, 8=32, 9=36, 10=40, at tolerances of ±15%. In some embodiments, amplitude to be nominally constant current (+/−200/% of output into 500-ohm load), is tested at 200 ohms and 1000-ohm loads. In some embodiments, amplitude may be automatically modulated using a 1 second ramp up to setting, 2 seconds constant at setting, 1 second ramp down to zero or lowest possible value, repeating continuously. In some embodiments, pulse width may be 350 μseconds+/−10%. In some embodiments, pulse frequency may be 31 Hz (32 msec period between alternating monophasic pulses)+/−10%. In some embodiments, pulse waveform may be such that the ratio of: [pulse amplitude at 50+/−2 μseconds after the start of the pulse] to [peak pulse amplitude at start of pulse] may be equal to 0.51+/−12%. In some embodiments, waveform will be such that the ratio of: [pulse amplitude at 100+/−4 μseconds after the start of the pulse] to [peak pulse amplitude at start of pulse] will be equal to 0.26+/−20%. In some embodiments, the device may incorporate an output regulation feature such that the stimulation output amplitude does not vary more than 20%/o from the nominal as the battery charge decreases while in use. In some embodiments, the device may incorporate an automatic shut off feature such that when output regulation cannot be maintained for any setting, the device will shut off. In some embodiments, battery capacity and performance will deliver a minimum of 16 hours of continuous therapy from a full charge at intensity level 5 (which is half of the maximum output, or 20 mA output level) when tested using a 500 ohm purely resistive load.

In some embodiments, the overall waveform comprises a series of bipolar trapezoidal waveform that make low frequency pulses. The waveform is initiated at low power levels of about 1 to 2 volts and ramps up over a period of about 1 second to a maximum level of 17-18 volts, and is maintained for about 2 seconds, and then ramps down over a period of about 1 second to low power levels of about 1 to 2 volts. (The voltage specified is measured across a 500 ohm resistive load as suggested in ANSI/AAMI NS-4 (1986), American National Standard for Transcutaneous Electrical Nerve Stimulators.) The individual pulses are separated by about 31-32 milliseconds (msec)(measured peak to peak), and last about 350 microseconds (μsec) at power levels of about 1-100 milliamps peak pulse height, in some embodiments preferably 10-50 milliamps peak pulse height and in some embodiments 10-40 milliamps peak pulse height. A wide range of pulse patterns may be used in noninvasive nerve stimulation devices. The individual pulses alternate between negative and positive pulses, and are said to constitute a bipolar waveform. The individual pulse is made of a sharply vertical spike which decays exponentially over a period of about 350 sec, thus comprising a basically vertical leading edge and an exponentially decaying trailing edge to each individual pulse. The following pulse will be shaped the same, except that it will be of negative voltage. The exponential nature of the individual pulse decay maximizes the high frequency components in the signal. These high frequency components contribute to a lessening of the skin impedance, in particular the capacitive components. This contributes to a higher level of current able to enter the deeper tissues. The power levels may be adjusted up or down to intensify the therapeutic effect of the device or lessen the sensation causes by the device, according to the preferences of individual users. The pulse rate within the waveform may be increased or decreased also.

To use one of the devices such as those set forth in U.S. Pat. Nos. 4,981,146, 6,076,018, 6,567,695, 6,735,480, 7,127,288, 7,171,266, 7,171,276, 7,983,761, the user merely secures the device on their wrist positioning the electrodes over the P6 acupuncture point, in electrical contact with the skin overlying the median nerve. The user then turns the device on, adjusts it to a comfortable power level, and allows stimulation to continue for a few minutes, for example 5-10 minute or longer, in some embodiments 30 minutes or longer, in some embodiments 30 minutes or longer, in some embodiments 90 minutes or longer, in some embodiments 120 minutes or longer, in some embodiments 150 minutes or longer, in some embodiments 180 minutes or longer, to achieve relief. The device may be applied intermittently, once every hour or so, or continuously. The device provides electrical current and voltage to the electrodes.

The present disclosure includes a configuration for an electrode and/or pair of electrodes which may be used in conjunction with an electrotherapy device. The electrodes are preferably made of 316L stainless steel and manufactured by metal injection molding. This material and process are used to produce electrodes which allow the sensation of the applied electrotherapy to be more tolerable to a greater number of users. It should be understood, however, that various features disclosed herein are exemplary and that other configurations and processes are possible.

1. A P6 electrostimulation device comprising a pair of electrodes connected to electronic control circuitry that comprises a power supply and pulse generation circuitry, wherein the electrodes are made of 316L stainless steel and manufactured by metal injection molding.

2. The P6 electrostimulation device of claim 1, wherein each of the electrodes is D shaped and has a straight edge and an arcuate edge, wherein the pair of electrodes are positioned on the device such that when the device is worn by a user and the electrodes are in contact with the P6 point on a user's wrist the straight edges of each opposing and parallel to each other and perpendicular with the direction of the user's arm between the user's wrist and elbow.

3. The P6 electrostimulation device of claim 2, wherein the straight edges have a length of about 22.00 to about 25.50 mm, and the arcuate edges having an inner radius of about 10.50 to about 13.00 mm, and a thickness of about 0.50 to about 1.75 mm.

4. The P6 electrostimulation device of claim 2, wherein the straight edges have a length of about 23.00 to about 24.50 mm, and the arcuate edges having an inner radius of about 11.00 to about 12.70 mm, and a thickness of about 0.80 to about 1.40 mm.

5. The P6 electrostimulation device of claim 2, wherein the straight edges have a length of about 23.84+/−0.10 mm and the arcuate edges having an inner radius of about 11.83+/−0.10 mm, and a thickness of about 1.08+/−0.10 mm.

6. The P6 electrostimulation device of claim 2, wherein the straight edges of the pair of electrodes face each other in apposition and are separated by a gap that is 0.05 to 0.5 inches or 1-15 mm.

7. The P6 electrostimulation device of claim 2, wherein the straight edges of the pair of electrodes face each other in apposition and are separated by a gap that is 0.1 to 0.25 inches or 2-7.5 mm.

8. The P6 electrostimulation device of claim 2, wherein the straight edges of the pair of electrodes face each other in apposition and are separated by a gap that is 3-5 mm.

9. The P6 electrostimulation device of claim 2, wherein the straight edges of the pair of electrodes face each other in apposition and are separated by a gap that is 0.14 inches.

10. The P6 electrostimulation device of claim 2, wherein the electrodes are connected to electrical conduits that supply electrical output from the electronic control circuitry at a position on or immediately adjacent to within 5 mm of the center point of the arcuate edge.

11. The P6 electrostimulation device of claim 2, wherein the electrodes are connected to electrical conduits that supply electrical output from the electronic control circuitry at a position on or immediately adjacent to within 3 mm of the center point of the arcuate edge.

12. A D shaped electrode made of 316L stainless steel and manufactured by metal injection molding, wherein the electrode has a straight edge having a length of about 22.00 to about 25.50 mm and an arcuate edge having an inner radius of about 10.50 to about 13.00 mm, and a thickness of about 0.50 to about 1.75 mm.

13. The D shaped electrode of claim 12, wherein the straight edge has a length of about 23.00 to about 24.50 mm, and the arcuate edge has an inner radius of about 11.00 to about 12.70 mm, and the electrode has a thickness of about 0.80 to about 1.40 mm.

14. The D shaped electrode of claim 12, wherein the straight edge has a length of about 23.84+/−0.10 mm, and the arcuate edge has an inner radius of about 11.83+/−0.10 mm, and the electrode has a thickness of about 1.08+/−0.10 mm.
